(19) United States
(12) Patent Application Publication
(10) Pub. No.: US 2012/0203317 A1
(43) Pub. Date: Aug. 9, 2012

(54) ENCLOSURE

(75) Inventors: Craig Valentine, Lancashire (GB); Nuno Sereno, Merseyside (GB); Marcus Jarman-Smith, Lancashire (GB)

(73) Assignee: INVIBIO LIMITED, Lancashire (GB)

(21) Appl. No.: 13/382,071

(22) PCT Filed: Jul. 2, 2010
(86) PCT No.: PCT/GB2010/051097

§ 371 Date: May 1, 2012

(30) Foreign Application Priority Data
Jul. 2, 2009 (GB) 0911489.3

(51) Int. Cl. A61N 1/36; B23P 11/00

(52) U.S. Cl. 607/116; 29/428

(57) ABSTRACT

An enclosure of an active implantable device comprises a composite material which comprises a first layer of a polyetheretherketone film, a second layer of polyetheretherketone film (8) and, sandwiched between, a third layer comprising titanium. The arrangement may be provided in various embodiments such as comprising housing halves (70,72) and a lid (74), each being made of the three layers described. In one embodiment, a communications window may be provided which has a thinner layer of titanium, to facilitate communications between the outside and electrical components positioned within the enclosure.

This invention relates to an enclosure. Preferred embodiments relate to an enclosure for an implantable device for implantation in a human body and a method of making such enclosures and devices.

It is well known to provide active implantable medical devices for implantation in a human body for applying a stimulus to a part of the body, for example a tissue, for a therapeutic purpose. Such implantable devices may be arranged to supply an electrical stimulus used in neurological therapy for stimulating nerves or muscle to combat pain or may be used as a heart pacemaker. Other applications include use in treating urinary urge incontinence by stimulating nerves close to the pelvic floor; and use in reduction of pressure sores by stimulating cavernous nerves. In addition, implantable devices are, in some cases, used to provide a chemical or mechanical stimulus.

Implantable devices may, in general terms, comprise an enclosure which includes electronic circuitry and a power source. Preferably, the enclosure defines an hermetically sealed environment so that its contents are protected from ingress of water which could be damaging.

Implantable devices are increasing in function and complexity. They may incorporate sensory loops between electrodes monitoring body/therapy behaviour and the communication of recorded data and control signals between the device and external systems. These signals are transmitted for example by means of radio frequency (RF) coupling. Moreover, devices are being developed which include batteries which are rechargeable by, for example, inductive coupling of power to a receiver coil.

The transmission of RF signals through an hermetic enclosure of an active implantable medical device may be affected by several factors, such as: a) the accuracy of the placement of the charging coil, b) the signal frequency, c) Eddy current losses in the housing, d) the charge rate of the battery and e) the Coulombic efficiency of the battery.

Current implantable devices are primarily enclosed in titanium alloy enclosure. However, a titanium alloy enclosure severely attenuates RF signals and generates increases in temperature due mainly to Eddy current losses associated with titanium material properties. Such effects mean that low operating frequencies have to be use and the battery recharging rate is decreased which has a detrimental effect on battery life.

It is an object of the present invention to address the above described problems. Preferred embodiments have the object of facilitating higher frequency telemetry communication with active implantable devices.

According to a first aspect of the invention, there is provided an enclosure which comprises a first region having a water vapour transmission rate of less than 1 g.m⁻².d⁻¹ and a second region outside the first region, wherein said second region comprises a second material having a Notched Izod Impact Strength of at least 1.0 KJ.m⁻².

Water Vapour Transmission Rates (WVTR) may be measured using DIN 53122 (25° C., 75% relative humidity). Notched Izod Impact Strength (NITS) may be measured, at 23°, as described in ASTM D256.

Said second region may comprise a said second material having a NITS of at least 2.0 kJm⁻², preferably at least 3.0 kJm⁻², more preferably at least 4.0 kJm⁻². The NITS may be less than 6.0 kJm⁻² or less than 5.0 kJm⁻².

Said second region is preferably arranged to provide the enclosure with appropriate impact strength to enable it to withstand normal forces to which it may be subjected in use. Said first region may comprise a first material which has an impact strength, for example a NIIS, of less than that of said second material.

Said second region is preferably a component of an enclosure wall of the enclosure, wherein said enclosure wall may be arranged to substantially fully enclose an internal volume in which components of an implantable device may be contained. Said second region suitably traverses at least 70%, preferably at least 90%, more preferably at least 95%, especially at least 98% of the area of said enclosure wall. Preferably, said first region substantially fully encloses said internal volume.

The thickness of the second region may be substantially constant over at least 30%, at least 50%, at least 80% or at least 90% of its area. In some areas, for example in a region of a communications window as described hereinafter, the thickness may vary.

Said second region may have a thickness of at least 100 μm Said second region may have a thickness of at least 100 μm over substantially its entire extent. Said second region may have a thickness of less than 1000 μm, preferably over substantially its entire extent.

Said second region preferably defines an outermost surface of the housing. Suitably, said second region defines at least 70%, preferably at least 90%, more preferably at least 95%, especially at least 99% of the area of the outermost surface of the housing.

Said second region preferably comprises a polymeric material which has a moiety of formula

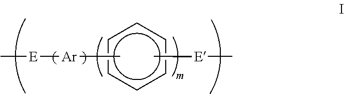

and/or a moiety of formula

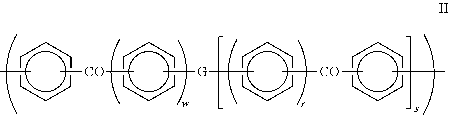

and/or a moiety of formula

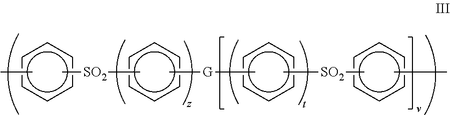

wherein m,r,s,t,v,w and z independently represent zero or a positive integer, E and E′ independently represent an oxygen or a sulphur atom or a direct link, G represents an oxygen or sulphur atom, a direct link or a —O-Ph-O— moiety where Ph represents a phenyl group and Ar is selected from one of the following moieties (i)**, (i) to (iv) which is bonded via one or more of its phenyl moieties to adjacent moieties

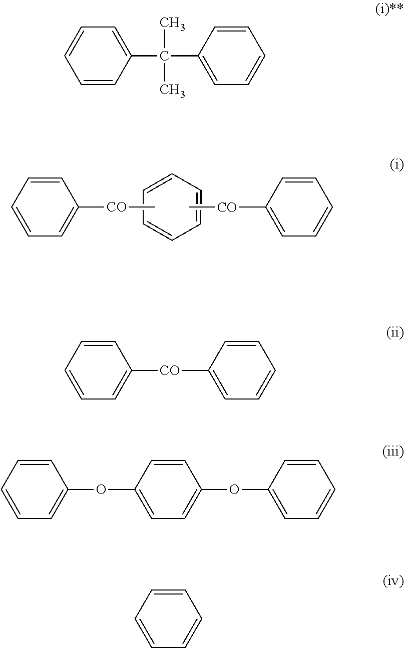

Unless otherwise stated in this specification, a phenyl moiety has 1,4-, linkages to moieties to which it is bonded.

In (i), the middle phenyl may be 1,4- or 1,3-substituted. It is preferably 1,4-substituted.

Said polymeric material may include more than one different type of repeat unit of formula I; and more than one different type of repeat unit of formula II; and more than one different type of repeat unit of formula III. Preferably, however, only one type of repeat unit of formula I, II and/or III is provided.

Said moieties I, II and III are suitably repeat units. In the polymeric material, units I, II and/or III are suitably bonded to one another—that is, with no other atoms or groups being bonded between units I, II and III.

Phenyl moieties in units I, II and III are preferably not substituted. Said phenyl moieties are preferably not cross-linked.

Where w and/or z is/are greater than zero, the respective phenylene moieties may independently have 1,4- or 1,3-linkages to the other moieties in the repeat units of formulae II and/or III. Preferably, said phenylene moieties have 1,4-linkages.

Preferably, the polymeric chain of the polymeric material does not include a —S— moiety. Preferably, G represents a direct link.

Suitably, “a” represents the mole % of units of formula I in said polymeric material, suitably wherein each unit I is the same; “b” represents the mole % of units of formula II in said polymeric material, suitably wherein each unit II is the same; and “c” represents the mole % of units of formula Ill in said polymeric material, suitably wherein each unit Ill is the same. Preferably, a is in the range 45-100, more preferably in the range 45-55, especially in the range 48-52. Preferably, the sum of b and c is in the range 0-55, more preferably in the range 45-55, especially in the range 48-52. Preferably, the ratio of a to the sum of b and c is in the range 0.9 to 1.1 and, more preferably, is about 1. Suitably, the sum of a, b and c is at least 90, preferably at least 95, more preferably at least 99, especially about 100. Preferably, said polymeric material consists essentially of moieties I, II and/or Ill.

Said polymeric material may be a homopolymer having a repeat unit of general formula

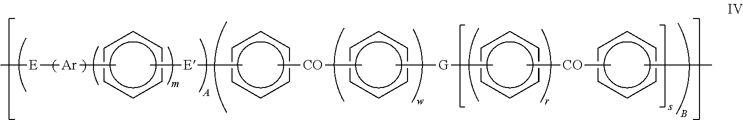

or a homopolymer having a repeat unit of general formula

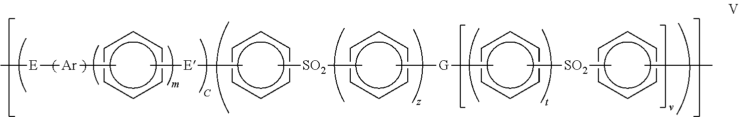

or a random or block copolymer of at least two different units of IV and/or V,wherein A, B, C and D independently represent 0 or 1 and E,E′,G,Ar,m,r,s,t,v,w and z are as described in any statement herein.

Preferably, m is in the range 0-3, more preferably 0-2, especially 0-1. Preferably, r is in the range 0-3, more preferably 0-2, especially 0-1. Preferably t is in the range 0-3, more preferably 0-2, especially 0-1. Preferably, s is 0 or 1. Preferably v is 0 or 1. Preferably, w is 0 or 1. Preferably z is 0 or 1.

Preferably, said polymeric material is a homopolymer having a repeat unit of general formula IV.

Preferably Ar is selected from the following moieties (xi)** and (vii) to (x)

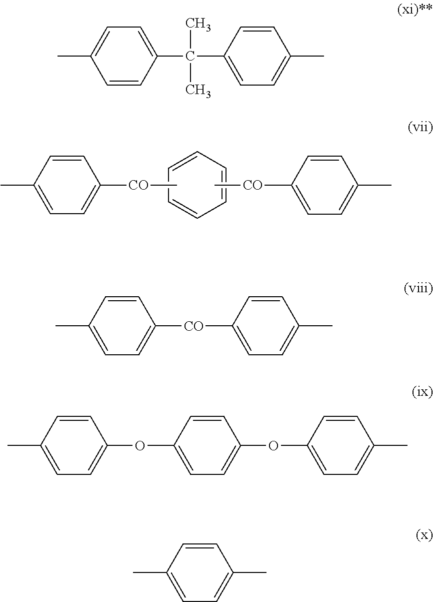

In (vii), the middle phenyl may be 1,4- or 1,3-substituted. It is preferably 1,4-substituted.

Suitable moieties Ar are moieties (i), (ii), (iii) and (iv) and, of these, moieties (i), (ii) and (iv) are preferred. Other preferred moieties Ar are moieties (vii), (viii), (ix) and (x) and, of these, moieties (vii), (viii) and (x) are especially preferred.

An especially preferred class of polymeric materials are polymers (or copolymers) which consist essentially of phenyl moieties in conjunction with ketone and/or ether moieties. That is, in the preferred class, said polymeric material does not include repeat units which include —S—, —SO₂— or aromatic groups other than phenyl. Preferred polymeric materials of the type described include:
(a) a polymeric material consisting essentially of units of formula IV wherein Ar represents moiety (iv), E and E′ represent oxygen atoms, m represents 0, w represents 1, G represents a direct link, s represents 0, and A and B represent 1 (i.e. polyetheretherketone).
(b) a polymeric material consisting essentially of units of formula IV wherein E represents an oxygen atom, E′ represents a direct link, Ar represents a moiety of structure (i), m represents 0, A represents 1, B represents 0 (i.e. polyetherketone);
(c) a polymeric material consisting essentially of units of formula IV wherein E represents an oxygen atom, Ar represents moiety (i), m represents 0, E′ represents a direct link, A represents 1, B represents 0, (i.e. polyetherketoneketone).
(d) a polymeric material consisting essentially of units of formula IV wherein Ar represents moiety (i), E and E′ represent oxygen atoms, G represents a direct link, m represents 0, w represents 1, r represents 0, s represents 1 and A and B represent 1. (i.e. polyetherketoneetherketoneketone).
(e) a polymeric material consisting essentially of units of formula IV, wherein Ar represents moiety (iv), E and E′ represents oxygen atoms, G represents a direct link, m represents 0, w represents 0, s, r, A and B represent 1 (i.e. polyetheretherketoneketone).
(f) a polymeric material comprising units of formula IV, wherein Ar represents moiety (iv), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said polymeric material may be amorphous or semi-crystalline. Said polymeric material is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning calorimetry (DSC).

The level of crystallinity in said polymeric material may be at least 1%, suitably at least 3%, preferably at least 5% and more preferably at least 10%. In especially preferred embodiments, the crystallinity may be greater than 30%, more preferably greater than 40%, especially greater than 45%.

The main peak of the melting endotherm (Tm) for said polymeric material (if crystalline) may be at least 300° C.

Said polymeric material may consist essentially of one of units (a) to (f) defined above.

Said polymeric material preferably comprises, more preferably consists essentially of, a repeat unit of formula (XX)

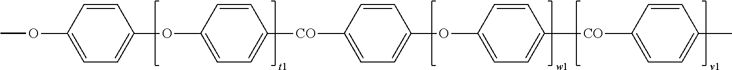

where t1, and w1 independently represent 0 or 1 and v1 represents 0, 1 or 2. Preferred polymeric materials have a said repeat unit wherein t1=1, v1=0 and w1=0; t1=0, v1=0 and w1=0; t1=0, w1=1, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said polymeric material suitably has a melt viscosity (MV) of at least 0.06 kNsm⁻², preferably has a MV of at least 0.085 kNsm⁻², more preferably at least 0.12 kNsm⁻², especially at least 0.14 kNsm⁻².

MV is suitably measured using capillary rheometry operating at 400° C. at a shear rate of 1000 s⁻¹ using a tungsten carbide die, 0.5×3.175 mm.

Said polymeric material may have a MV of less than 1.00 kNsm⁻², preferably less than 0.5 kNsm⁻².

Said polymeric material may have a MV in the range 0.09 to 0.5 kNsm⁻², preferably in the range 0.14 to 0.5 kNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said second region suitably comprises at least 60 wt %, preferably at least 70wt %, more preferably at least 80wt %, especially of at least 90wt % of a thermoplastic polymeric material, for example a said polymeric material described above for example of formula (XX), especially polyetheretherketone. Said second region preferably consists essentially of a said polymeric material, especially polyetheretherketone.

Said second region may have a WVTR of less than the WVTR of the first region. The ratio of the WVTR of the first region to that of the second region may be at least 10, at least 100, at least 1000 or at least 10000.

Said first region is preferably arranged to electromagnetically shield an internal volume of the housing so as to reduce the effects of electromagnetic fields emanating outside the housing on electronic components contained within the housing. Additionally or alternatively, said first region is preferably arranged to substantially hermetically seal the housing thereby to substantially limit the passage of moisture into the internal volume of the housing.

Said first region may have a WVTR of less than 1×10⁻¹ g.m⁻².d⁻¹, preferably less than 1×10⁻² g.m⁻².d⁻¹, more preferably less than 1×10⁻³g.m⁻².d⁻¹, especially less than 1×10⁻⁴g.m⁻².d⁻¹. Said first region may be arranged to render the enclosure hermetic for a period of at least 1 year, 5 years, 10 years or 20 years when in situ in a human body.

Said first region is preferably a component of an enclosure wall of the enclosure, wherein said enclosure wall may be arranged to substantially fully enclose an internal volume in which components of an implantable device may be contained. Said first region suitably traverses at least 70%, preferably at least 90%, more preferably at least 95%, especially at least 98% of the area of said enclosure wall. Preferably, said first region substantially fully encloses said internal volume.

The thickness of the first region may be substantially constant over at least 50%, more preferably at least 80%, especially at least 90%, of its area. In some cases, for example in a region of a communications window as described hereinafter, the thickness may vary slightly.

Said first region may have a thickness of at least 1 μm, suitably at least 10 μm, preferably at least 20 μm, more preferably at least 50 μm, especially at least 80 μm Said first region may have a thickness of at least 1 μm, 3 μm or 7 μm over substantially its entire extent. Said first region may have a thickness of less than 200 μm, less than 150 μm or less than 100 μm.

Said first region may comprise or consist essentially of a metal, ceramic or plastics material. A metal may be a metal alloy; it may be aluminium or titanium with the latter being especially preferred. Said first region preferably comprises titanium. It preferably comprises a layer of titanium. Said first region preferably consists essentially of titanium.

The distance between the first and second regions may be less than 10 μm or less than 5 μm. Said first and second regions may abut and/or make face to face contact. Alternatively, an adhesive layer may be provided between the first and second regions.

Said first and second regions are preferably defined by first and second layers of the materials described.

Said enclosure preferably includes a third region inwardly of the second region and preferably inwardly of the first region. Said third region is preferably arranged to electronically insulate components of the enclosure which may, in use, be contained within an internal volume of the device from other parts of the enclosure, for example from the first region which, in a preferred embodiment, comprises titanium which is an electrical conductor.

Said third region is preferably a component of an enclosure wall of the enclosure, wherein said enclosure wall may be arranged to substantially fully enclose an internal volume in which components of an implantable device may be contained. Said third region suitably traverses at least 70%, preferably at least 90%, more preferably at least 95%, especially at least 98% of the area of said enclosure wall. Preferably, said third region substantially fully encloses said internal volume.

The thickness of the third region may be substantially constant over at least 80%, preferably at least 90% of its area. In some areas, for example in a region of a communications window as described hereinafter, the thickness may vary slightly.

Said third region may have a thickness of at least 10 μm. Said third region may have a thickness of at least 50 μm over substantially its entire extent. Said third region may have a thickness of less than 500 μm, preferably less than 300 μm, more preferably less than 200 μm. Said third region preferably defines an inwardly facing surface of the housing. Suitably, said third region defines at least 70%, preferably at least 90%, more preferably at least 95%, especially at least 99% of the area of the inwardly facing surface of the housing.

Said third region preferably comprises a thermoplastic polymeric material. It may independently comprise a polymeric material comprising moieties I, II and/or III as described above and include the preferred features of said polymeric material described. It preferably comprises a repeat unit of formula (XX), especially polyetheretherketone. Said third region preferably consists essentially of polyetheretherketone.

The third region is suitably closer to the first region than to the third region. Said first region preferably spaces the second and third regions from one another.

The distance between the first and third regions may be less than 10 μm or less than 5 μm. Said first and third regions may abut and/or make face to face contact. Alternatively, an adhesive layer may be provided between first and third regions.

Said enclosure preferably comprises a second region which comprises a second layer of material described, a first region inwards of the second region which comprises a second layer of material described, and a third region inwards of both the first and second regions, wherein the third region comprises a third layer of material described.

The enclosure may include a communications window which suitably comprises a region of the enclosure which is arranged to more readily transmit electromagnetic radiation between a position outside the housing and electronic components which may be provided within the housing, for example to facilitate the recharging of a battery within the housing or the passage of control signals or information between the housing and apparatus, for example a monitor, spaced from the housing. Said communications window may be defined, in part, by a communications area of the first region which is thinner (and therefore more transmissive of electromagnetic radiation) than areas of the first region which surround the communications area. The ratio of the thickness of the first region in the region of the communications area to the thickness outside the area may be in the range 0.1 to 1, suitably 0.2 to 0.8, preferably 0.2 to 0.6, more preferably 0.3 to 0.5. The thickness of the first region in the region of the communications area may be less than 200 μm, preferably less than 150 μm, more preferably less than 120 μm. The thickness may be at least 0.5 μm, at least 1 μm, at least 5 μm or at least 10 μm. The thickness of the first region outside the area of the communications window may be at least 20 μm, preferably at least 50 μm, more preferably at least 100 μm.

The thickness of the second region in the region of the communications area may be greater than the thickness of the second region in areas of the second region which surround the communications area, for example to compensate for the reduction in thickness of the first region in the region of the communications area. The ratios of the thickness of the second region in the region of the communications area to the thickness in areas of the second region which surround the communications area is preferably at least 1.05, more preferably at least 1.1, especially at least 1.2.

Said enclosure may comprise a plurality of parts which are secured to one another. Each of said parts preferably includes first, second and optional third regions described herein. A hermetic seal is preferably defined between adjacent respective first regions of the plurality of parts, for example by welding. Thus, first regions of one part of the enclosure are preferably secured to first regions of another part of the enclosure by welding. A continuous, uninterrupted hermetic seal is preferably defined between said plurality of parts of the enclosure.

According to a second aspect of the invention, there is provided an implantable device which comprises an enclosure according to the first aspect.

Said implantable device is suitably for implantation in a human body. It is preferably an active implantable device which is suitably arranged to apply a stimulus to part of the body, for example for therapeutic purposes. The device may be arranged to apply an electrical stimulus.

Said implantable device may include a communications device for communicating information to a position outside the device.

Said implantable device may include a battery, for example a rechargeable battery. The battery may be arranged to be recharged by supply of energy through the atmosphere to the device so that recharging does not consist of or include the supply of energy via electrical wiring between the device and a supply of energy for recharging.

According to a third aspect of the invention, there is provided a layered structure comprising:

a first region according to the first aspect;

a second region according to the first aspect; and

a third region according to the first aspect.

The first, second and third regions of the third aspect may have any features of the first, second and third regions of the first aspect mutatis mutandis.

In a preferred embodiment, said first region comprises titanium, said second region comprises a polymeric material of formula (XX), especially polyetheretherketone, and said third region comprises a polymeric material of formula (XX), especially polyetheretherketone. The first region may have a thickness in the range 1 μm to 200 μm, said second region may have a thickness in the range 100 μm to 1000 μm and said third region may have a thickness in the range 10 μm to 500 μm.

According to a fourth aspect of the invention, there is provided the use of an implantable device according to the second aspect for implantation into a human body for applying a stimulus to the body.

According to a fifth aspect of the invention, there is provided a method of treating a condition of a human body comprising:
selecting an implantable device according to the second aspect;
implanting the implantable device into the human body, wherein the implantable device is arranged to apply a stimulus to the body to treat the condition.

According to a sixth aspect, there is provided a method of making an implantable device according to the first aspect, the method comprising selecting an enclosure or parts thereof of the first aspect and associating means for applying a stimulus to a part of a human body with the enclosure or parts thereof. The method suitably comprises arranging the enclosure so that it is substantially hermetic.

According to a seventh aspect, there is provided a method of making a layered structure according to the third aspect, the method comprising associating means to define the first, second and third regions with one another.

Any feature of any aspect of any invention or embodiment described herein may be combined with any feature of any aspect of any other invention or embodiment described herein mutatis mutandis.

Specific embodiments of the invention will now be described, by way of example, with reference to the accompanying drawings, in which:

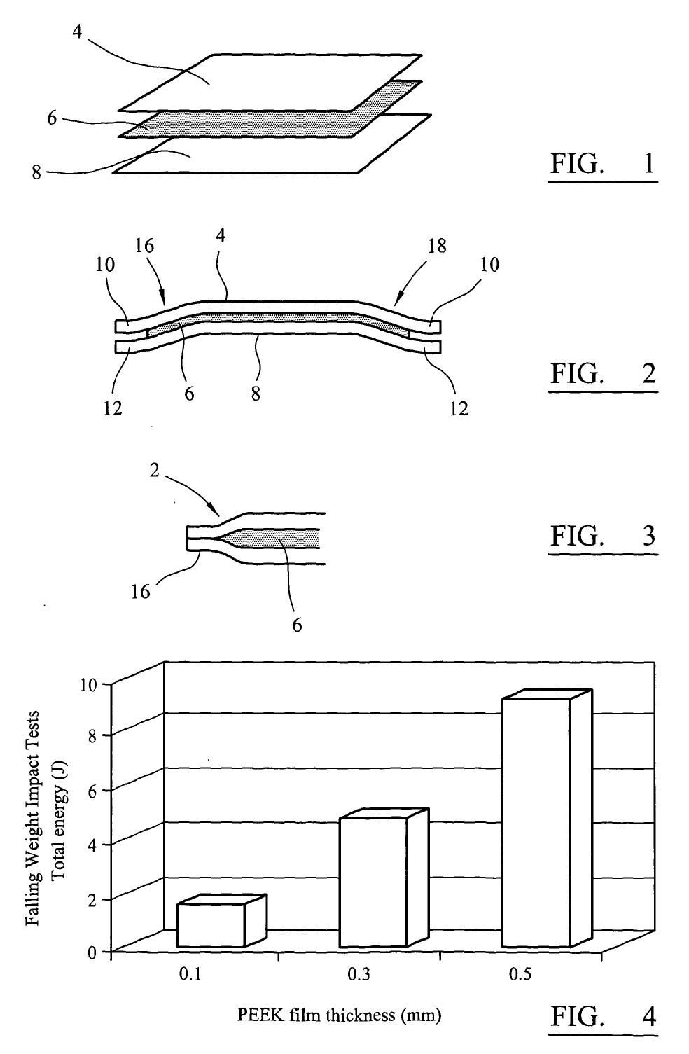

FIG. 1 is an exploded perspective view showing layers of a composite material for use in making an active implantable medical device;

FIG. 2 is a side view of a composite material during its manufacture;

FIG. 3 is a side view of an end of the composite material of FIG. 2 after further treatment;

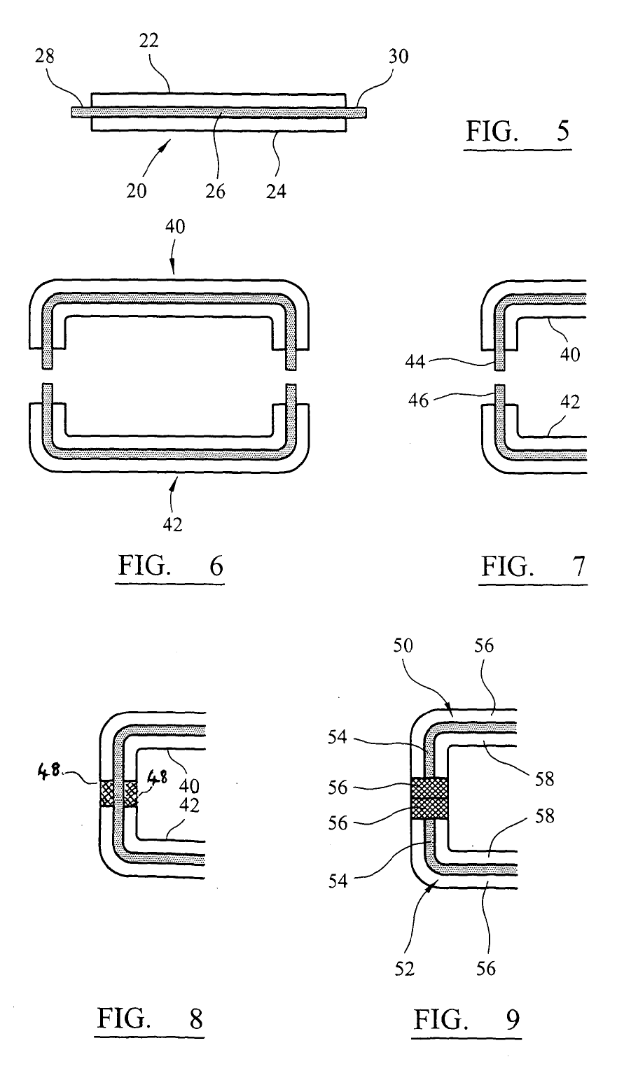

FIG. 4 illustrates the impact strength of polyetheretherketone at three different film thicknesses;

FIG. 5 is a side view of an alternative composite material;

FIG. 6 is a side view showing two housing halves thermoformed out of the material of FIG. 5 being presented to one another prior to forming a housing;

FIG. 7 is an enlarged view showing edges of the housing halves being presented to one another;

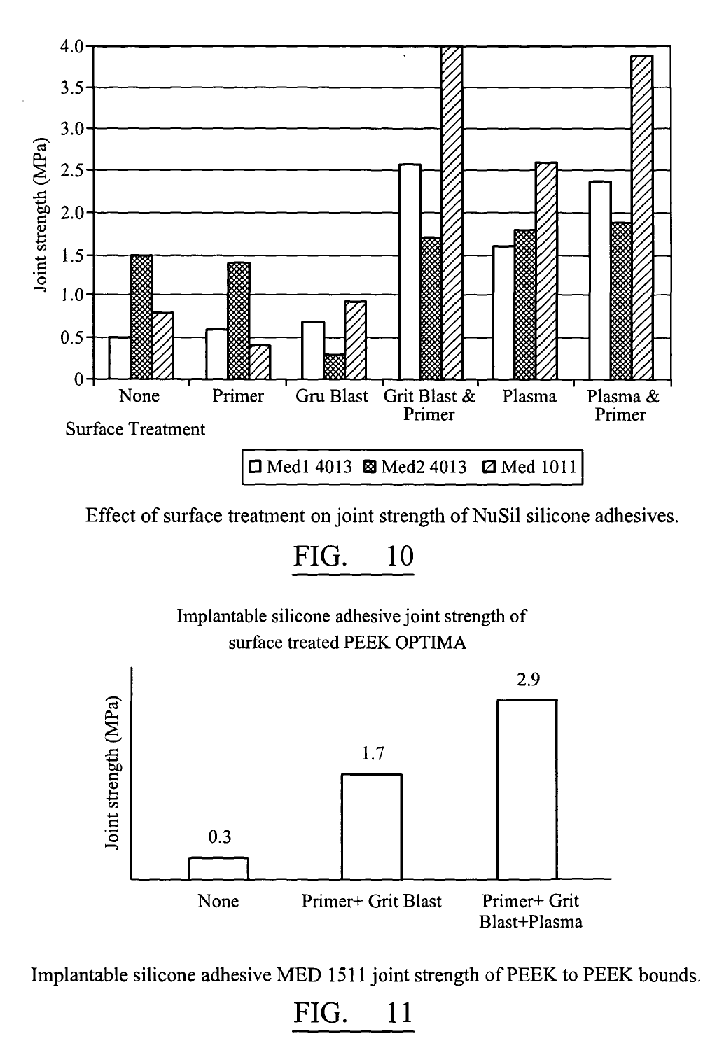

FIG. 8 shows edges of two housing halves secured to one another;

FIG. 9 shows an alternative arrangement of titanium layers at opposing edges of housing halves;

FIG. 10 is a representation illustrating the effect of surface treatment on joint strength of specified silicone adhesives;

FIG. 11 is a representation similar to FIG. 10;

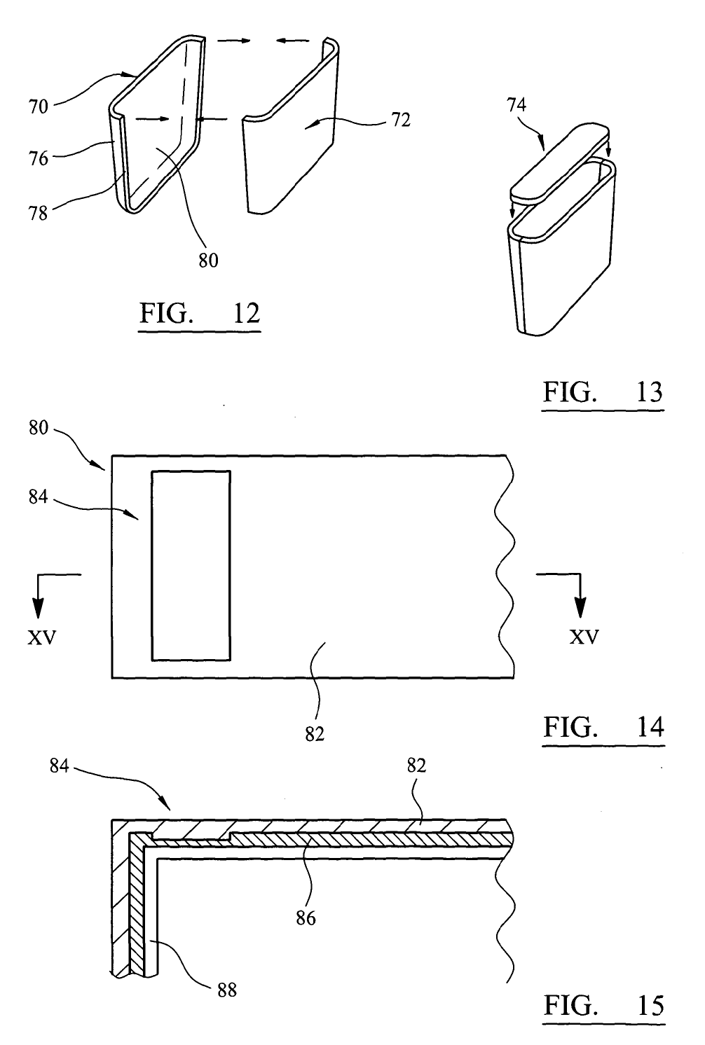

FIG. 12 shows two housing halves being secured to one another;

FIG. 13 shows a lid being secured to the assembly of FIG. 12.

FIG. 14 is a plan view of a housing having a communication window; and

FIG. 15 is a partial cross-section along line XV-XV of FIG. 14.

The following materials are referred to herein

PEEK-OPTIMA—referas to polyetheretherketone obtained from Invibio Ltd.

MED1511—an implantable silicone adhesive from Nusil

Referring to FIGS. 1 to 3, a composite material 2 for use in making an active implantable medical device comprises a first layer of a polyetheretherketone film 4, a second layer of polyetheretherketone film 8 and, sandwiched therebetween, a third layer comprising titanium 6. The first, second and third layers have the same width but the length of the titanium layer may be slightly less than that of the first and second polyetheretherketone layers. In other embodiments, the length of the titanium layer may be slightly greater than that of the polyetheretheketone layers so titanium protrudes beyond opposing edges defined by polyetheretherketone layers.

The first, second and third layers are secured to one another by heat press welding using a temperature of about 400° C., a pressure of 1 to 10 bar and a welding time of 10 to 180 seconds. Alternatively or additionally the layers may be secured by means of adhesives (e.g. silicone, epoxy or cyanoacrylate adhesives). Bonding may be enhanced by prior surface treatment of the layers for example by grit blasting, chemical etching or by treatment with a plasma, corona, laser or UV light Mechanical surface roughening of the surface may be accomplished using silicon carbide or by sand or mechanical roughening. The surfaces should be first degreased with MEK or acetone, roughened and then cleaned again in order to remove debris and grease. Chemical etching of carbon fibre filled PEEK surfaces has been achieved using a composition of K₂Cr₂O₇, H₂O and H₂SO₄, as described in Davies, P., et al., Surface treatment for adhesive bonding on carbon fibre-poly(etheretherkethone) composites. Journal of Materials Science Letters, 1991(10): p. 335-338. Cold gas plasma treatment imparts surface modification by altering the surface chemistry of a polymer and, if carried out long enough, will also have an effect on surface roughening. Typical gases used for the treatment of polymers are air, oxygen, nitrogen, helium, argon and ammonia. Corona treatment utilises a glow discharge similar to plasma treatment but operating in air and at atmospheric pressure. Laser treatment of a material surface is accomplished by exciting either gas or a solid to emit light of a particular wavelength. This energy chemically modifies the surface and promotes surface roughening or ablation. UV-light treatment involves delivering light at wavelengths between 172 nm and 308 nm to alter the surface of a material

After securement of the first, second and third layers to one another, the structure shown in FIG. 2 may be formed wherein the layers are bonded to one another with the titanium layer 6 slightly inwards of the outer edges of the polyetheretherketone layers 4. To complete the composite material, superimposed polyetheretherketone areas 10, 12 at opposing ends of the material are heat pressed together (temperature 400° C., pressure 1 to 10 bar and welding time 10 to 180 seconds) to define a structure illustrated in FIG. 3 wherein the titanium layer 6 is fully enclosed by the polyetheretherketone layers along the extent of respective edges 16, 18 of the material.

The composite material prepared can be used to make a housing of an active implantable medical device, for example by thermoforming. Where a shape formed needs to be bonded to another part in a hermetic manner, the titanium layer may be exposed and a titanium to titanium bond formed, for example by laser welding, between two composite materials of the type described. Alternatively, composite materials as described hereinafter with reference to FIGS. 5 to 8 may be used.

The thicknesses of the first, second and third layers may be selected according to the requirements of any particular situation. Suitably, a polyetheretherketone layer which is to be an outer layer in use is arranged to provide impact strength; the titanium layer is arranged to provide hermeticity; and the polyetheretherketone layer which is an inner layer in use is arranged to provide electrical insulation. Furthermore, the arrangement of layers allows for improved RF telemetry with reduced heating resulting from Eddy current losses. the polyetheretherketone layers are selected to provide strength to the composite material and/or to be able to withstand sterilisation.

A preferred polyetherethereketone is PEEK-OPTIMA (Trade Mark) which is a safe, biocompatible and stable polymer. PEEK-OPTIMA® has been extensively tested to ISO 10993 standards and demonstrated no evidence of cytotoxicity, systematic toxicity or irritation. PEEK-OPTIMA® polymer can be repeatedly sterilized using conventional sterilization methods including steam, gamma radiation and ethylene oxide processes without the degradation of its mechanical properties or biocompatibility. PEEK-OPTIMA® polymer is naturally radiolucent and compatible to imaging techniques such as X-ray, MRI and Computer Tomography (CT). The mechanical properties of PEEK-OPTIMA (Table 1) allow for it to meet the physical demands of an AIMD enclosure under selected thickness values.

Physical properties of PEEK-OPTIMA are provided in the table below.

| Property | Method | Value |
|---|---|---|
| Mechanical properties | | |
| Density (g · cm⁻³) | ASTM D792 | 1.3 × 10⁺⁰⁰ |
| Tensile strength (MPa) | ISO 527 Type 1B at | 101 |
| | 50 mm · min⁻¹ | |
| Elastic modulus (GPa) | ASTM D638 TV | 3.5 |
| Elongation at break (%) | ISO 527 Type 1B at | 20-30 |
| | 50 mm · min⁻¹ | |
| Flexural strength (MPa) | ISO 178 | 174 |
| Flexural modulus (GPa) | ISO 178 | 4.2 |
| Izod Notched Impact (kJ · m⁻²) | ASTM D256 | 4.3 |
| Glass transition temperature (° C.) | DSC | 142 |
| Melt temperature (° C.) | DSC | 344 |
| Specific heat capacity (KJ · Kg⁻¹ · ° C.⁻¹) | DSC | 2.16 |
| Thermal conductivity coefficient | ASTM C177 | 2.5 × 10⁻⁰¹ |

Furthermore, PEEK-OPTIMA is beige under translucent skin which may be highly relevant to, for example, cranial implants. Additionally, the polymer will not conduct heat nor cold which can cause discomfort.

The impact strength of films of polyetheretherketone which may be used in layers of the composite material 2 have been tested under ASTM D3763 to confirm that properties are suitable for use in medical devices described. The results of falling weight impact tests for film thickness of 0.1 mm, 0.3 mm as 0.5 mm are shown in FIG. 4.

Furthermore, the electrical insulation properties of the polyetheretherketone, detailed in Table 2, are such that it may advantageously be used in medical devices in the manner described.

TABLE 2
PEEK electrical properties (23° C., 1bar, 100 μm film

| Electrical properties | Conditions | PEEK |
|---|---|---|
| Conductivity (S · m⁻¹) | — | 1.50 × 10⁻¹⁵ |
| Dielectric strength (KV · mm⁻¹) | — | 1.98 × 10⁺⁰² |
| Breakdown voltage (thickness 50 μm, kV) | | 9.5 |
| Dissipation factor | 1 KH | 2 × 10⁻⁰³ |
| Volume resistivity (Ω · cm) | — | 4.9 × 10⁺¹⁶ |

The titanium layer thickness may be selected to make the composite material and/or a housing made therefrom substantially hermetic; but to present electrical properties which are not significantly detrimental to the functioning of an active implantable medical device which incorporates the composite material.

To establish the thickness of a titanium layer required to limit water ingress into a medical device to such an extent that the inside of the device will not become saturated with water vapour over the lifetime of the device, water vapour transmission rates (WVTR) were tested (3TC, RH 90%, pressure 1 bar) and results are provided in Table 3. Films of polyetheretherketone display permeability values to water of the same magnitude as observed for other high performance thermoplastic polymers.

TABLE 3
Water vapour transmission rate (WVTR) of tested systems.
One sided diffusion of water into plane film sheets
(temperature 37° C., RH 90%, pressure 1bar).

| | | Water vapour transmission rate |
|---|---|---|
| | Film thickness | (WVTR) |
| Sample | (μm) | (g · m⁻² · d⁻¹) |
| Titanium | 1 | 1.4 × 10⁻⁰⁵ |
| | 100 | <1 × 10⁻⁰⁶ |

It has been concluded from the aforesaid values that a combination comprising a polyetheretherketone layer with a 10 μm or thicker layer of titanium will create a water vapour impermeable barrier for the lifespan of an active implantable medical device.

Although polyetheretherketone has reasonable resistance to gas permeation a composite comprising polyetheretherketone and titanium can be used to achieve exceptionally low gas permeation values having regard to the data shown in Table 4.

TABLE 4
Gas permeation coefficients of PEEK and Titanium (film
thickness 100 μm, temperature 37° C., RH 90%, pressure 1bar).
Gas permeation coefficients (cm³ · m⁻² · d⁻¹)

| | PEEK | |
|---|---|---|
| Test gas | polyetheretherketone | Titanium |
| Carbon Dioxide | 4.24 × 10⁺⁰² | — |
| Helium | 1.57 × 10⁺⁰³ | <7.4 × 10⁻⁰³ (at 23° c.) |
| Hydrogen | 1.43 × 10⁺⁰³ | — |
| Nitrogen | 1.5 × 10⁺⁰¹ | — |
| Oxygen | 7.6 × 10⁺⁰¹ | — |

Additionally, a composite material comprising a titanium layer is substantially impermeable to sodium ions as illustrated in Table 5, which is important in the context of any device which may be implanted.

TABLE 5
Sodium permeability coefficients
(temperature 37° C., RH 90%, pressure 1bar).

| | Film thickness | Sodium permeability |
|---|---|---|
| Sample | (μm) | (g · m⁻² · d⁻¹) |
| PEEK | 100 | ≈3 × 10⁻⁰¹ |
| Titanium | 100 | ≈0 |

Although titanium has some advantageous properties it has disadvantageous electromagnetic compatibility (EMC) properties in general and in comparison to polyetheretherketone. As a result, titanium housings have detrimental telemetry characteristics when used for medical devices which are arranged to communicate and/or interact with electrical and/or magnetic fields outside the device. By way of example, as shown in Table 6, a titanium layer of thickness 300 μm would provide a reduction in the electrical field magnitude and energy density of more than 99% for a signal frequency greater than 10 MHz. In contrast, polyetheretherketone has favourable EMC properties, as illustrated in Tables 6 and 7.

TABLE 6
PEEK and Titanium EMC electrical attenuation
behaviour (23° C., 1bar).

| | Skin depth (m) | |
|---|---|---|
| Conditions | PEEK | Titanium |
| 1 MHz | ≈1 × 10¹² | 3.70 ×10⁻⁰⁴ |
| 10 MHz | >>1 | 1.17 ×10⁻⁰⁴ |
| 100 MHz | >>1 | 3.70 ×10⁻⁰⁵ |
| 400 MHz | >>1 | 1.85 ×10⁻⁰⁵ |
| 1000 MHz | >>1 | 1.17 ×10⁻⁰⁵ |

TABLE 7
Amount of power lost by electromagnetic
waves traversing through PEEK and Titanium
(signal frequency 400 MHz, Temp 23° C., pressure 1bar).

| | Reduction in the electric field | | Reduction in the electric field | |
|---|---|---|---|---|
| Thickness | magnitude (%) | | energy density (%) | |
| (μm) | PEEK | Titanium | PEEK | Titanium |
| 18.5 | ≈0 | 63.2 | ≈0 | 86.5 |
| 100 | ≈0 | >99.3 | ≈0 | >99.7 |

Referring to table 7, considering a titanium layer with a thickness equal to the skin depth, the electric field magnitude is reduced to 36.8% of its incident value and the electric field energy density is attenuated to 13.5% of its initial value.

Compared to current devices which may operate at frequencies of less than 150KHz due to the thickness of titanium used, arrangements as described herein may allow higher frequencies, for example up to 400 MHz or 800 MHz to be used

Another electrical property of titanium which is disadvantageous is its influence on attempts to induction charge batteries contained within implantable devices. Table 8 includes calculations on the respective influences of polyetheretherketone and titanium on induction charging, on the basis of implantable battery characteristics displayed in Table 9 and a 15 mm radial enclosure.

TABLE 8
Influence of PEEK and Titanium on implantable
battery recharging

| Characteristics | PEEK | Titanium |
|---|---|---|
| Eddy current loss (mW) | ≈0 | 2.0 × 10⁺⁰² |
| Induction heating of casing (° C.) | ≈0 | 4.84 × 10⁺⁰⁰ |
| Battery loss to heating (mW) | 3.2 × 10⁺⁰⁰ | 3.2 × 10⁺⁰⁰ |
| Battery heating (° C.) | 7.8 × 10⁻⁰¹ | 7.8 × 10⁻⁰¹ |

TABLE 9
Implantable battery characteristics.

| Battery property | Value |
|---|---|
| Battery capacity (mAh) | 1.6 × 10⁺⁰² |
| Charge rate of a medical implantable Li battery (mAh) | 8.0 × 10⁺⁰¹ |
| Peak charging voltage (V) | 4 × 10⁺⁰⁰ |
| Charging power (mW) | 3.2 × 10⁺⁰² |
| Coulombic efficiency (%) | 9.0 × 10⁺⁰¹ |

It will be noted from Table 8 that, whereas titanium exhibits significant Eddy current losses and a heat rise in the casing, polyetheretherketone advantageously has a negligible effect on such properties.

An alternative to the arrangement of FIGS. 1 to 3 is illustrated in FIGS. 5 to 8.

Referring to FIG. 5, a composite material 20 comprises a first layer of a polyetheretherketone film 22, a second layer of polyetheretherketone film 24 and sandwiched in between a third layer comprising titanium 26. In contrast to the arrangement of FIGS. 1 to 3, the titanium layer 26 protrudes from the polyetheretherketone layers 22, 24 at respective ends 28, 30. This arrangement may facilitate the production of a hermetic seal between two housing halves 40, 42 (FIG. 6) which may be thermoformed from the material 20. In this regard, referring to FIG. 7, free edges 44, 46 of respective housing halves may be juxtaposed and then the exposed titanium edges of each half can be laser welded to one another so as to define a housing with a continuous uninterrupted cylindrical wall. As shown in FIG. 8, a gap between the polyetheretherketone layers may be filled with filler 48 (e.g. of epoxy or silicone resin).

A further alternative is shown in FIG. 9. In this case titanium layers 50, 52 each include a narrow portion 54 and, at the free edges, a wider portion 56, wherein the wider portions 56 have a width which is the same as the sum of the widths of the two polyetheretherketone layers 56, 58 and the titanium layer therebetween. Accordingly when housing halves are joined, there is no area that needs filling, in contrast to the FIG. 8 embodiment.

In one embodiment, a hermetic joint may be defined by combining female structures of FIG. 2 and male structures of FIG. 5. The male and female structures may be engaged and welded so a continuum comprising titanium extends between the two structures.

In further embodiments, the titanium sheet may be graduated so that it is not a constant thickness. Thus, it may be of one thickness suitable for improved telemetry characteristics when bonded between PEEK sheets, but may be a different thickness when exposed at the extremities for laser bonding.

A housing may be made from a composite material comprising one polyetheretherketone and one titanium layer, or could have more layers.

Layers could alternate and vary in thicknesses.

In some cases, titanium may be provided as an outer layer and this may be beneficial if the bone bonding advantages of titanium are desired. However, if an implant is to be located at a site of soft tissue and/or may need to be removed in the future, then it is preferred that the outer surface of the housing be composed of PEEK.

As described above, titanium areas can be laser welded to other titanium areas. Several alternative methods may be used to join polyetheretherketone to other materials and/or to itself as follows:

(i) fusion welding—PEEK regions having mm thicknesses can be welded to other similar regions using fusion welding to produce welds with a bond strength value of 52 MPa. The welding generates acceptable levels of heat build up that are unlikely to damage heat sensitive components, for example, on a circuit board. X-ray analysis of the welds has revealed that consistent joining can be achieved.

(ii) Direct through-transmission laser welding of PEEK can be successfully achieved for 0.25-0.50 mm thick layers of natural (unfilled) PEEK against black (carbon black filled) PEEK. Hermetic welding can be achieved.

(iii) Hot plate welding of PEEK to titanium can be successfully achieved with prior treatment of the titanium surface by grit blasting. Under lap shear test, the PEEK/titanium displayed a joint strength of 0.1 MPa.

(iv) Depending on the location of an interface, adhesives such as epoxy or cyanoacrylate may be used. The surface of the materials (PEEK to PEEK, or PEEK to other material) can be further treated to enhance the bonding. Comprehensive studies evaluated the performance of different adhesives, the impact of pre-surface treatment and temperature on PEEK joint strength. Some results are provided in FIG. 10.

As can be observed in FIG. 10 surface preparation techniques greatly improve PEEK to PEEK joint strength for a range of joining methodologies and medical grade silicone adhesives (MED 1-4013, MED 2-4013, MED 1011, Nusil Technologies, California, USA). Moreover combining surface treatments can have a synergistic effect as can be observed from FIG. 11 which provides results for the joint strength using Nusil MED-1511 bonding of untreated PEEK; PEEK primed and grit blasted and PEEK primed, grit blasted and subjected to a plasma treatment. NuSil MED-1511 from NuSil Technology is a one component silicone adhesive. It contains no solvents or plasticizer and cures at room temperature to form a silicone rubber. Consequently properties are high elasticity at moderate strength only at ambient temperature. It is USP-VI classified and therefore suitable for medical applications. It withstands sterilisation with ethylene oxide, dry heat or steam autoclaving. The highest joint strength observed during testing achieved for the use of adhesive bounding of PEEK on PEEK was 5.9 MPa (Mean Failure Load 1885N). This was achieved with Loctite 4035 (One-part cyanoacrylate adhesive). Joint design is an important factor for device bond strength. Tensile strength can be increased by increasing the width of the lap shear joint.

Before and after sterilization (steam, EtO and gamma sterilisation) the joint strength of PEEK to PEEK joints with MED1511 adhesive remained unchanged.

The adhesion of PEEK to metals has been proven to generate strong bounds. Tests with implantable silicone adhesive (e.g. MED1511) registered joint strength values for PEEK to titanium of 1.7 MPa and PEEK to CoCr of 1.7 MPa. The metals surface was pre-treated by grit blasting.

An enclosure of an implantable device may be as shown in FIGS. 12 and 13. It may comprise housing halves 70, 72 and a lid 74. Each structure 70, 72, 74 is of a PEEK (76)-titanium (78)-PEEK (80) sandwich construction with, in each case, titanium projecting from the PEEK layers to enable hermetic joints to be formed by welding titanium to titanium at the interfaces of structures 70, 72, 74.

A preferred embodiment of an enclosure is shown in FIGS. 14 and 15. The enclosure 80 of an active implantable medical device includes an outer layer 82 which is made from PEEK, a middle layer 86 of titanium and an inner layer 88 of PEEK. In the region of the communications window 84 the titanium has a thickness of about 10 μm whereas in regions outside the communications window the titanium thickness is about 100 μm. To compensate for the reduced thickness of titanium, the PEEK layer 82 in the region of the window 84 has an increased thickness. As an alternative, the thickness of layer 82 could be constant and the thickness of layer 88 could be increased in the region of the communications window. A communications window 84 is provided above a communications device (e.g. comprising a coil) to facilitate passage of communication signals from a position outside the device to a position below the window 84 within the device.

The outer layer 82 may have a thickness (outside the region of the communications window) of about 300 μm which will provide the outer surface of the housing with substantial impact resistance. By providing a thinner titanium layer in the region of the window, communications signals can enter the housing and operate the communications device provided in the housing. By providing a thicker titanium layer in the regions outside the window, electronics within the housing may be shielded from electronic interference emanating from outside the housing. The inner layer 88 may have a thickness of about 100 μm and may be provided to electronically insulate the electronics with the housing from the titanium layer.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An enclosure which comprises a first region having a water vapour transmission rate of less than 1 g.m⁻².d⁻¹ and a second region outside the first region, wherein said second region comprises a second material having a Notched Izod Impact Strength of at least 1.0 KJ.m⁻².

2. An enclosure according to claim 1, wherein said second region is a component of an enclosure wall of the enclosure wherein said enclosure wall is arranged to substantially fully enclose an internal volume in which components of an implantable device may be contained.

3. An enclosure according to claim 1, wherein said second region has a thickness of at least 100 μm and a thickness of less than 1000 μm.

4. An enclosure according to claim 1, wherein second region comprises a polymeric material which has a moiety of formula 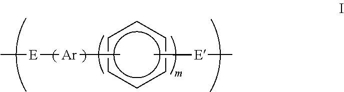
and/or a moiety of formula 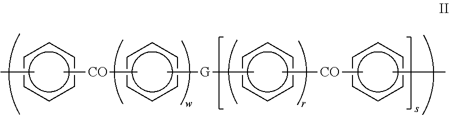
and/or a moiety of formula 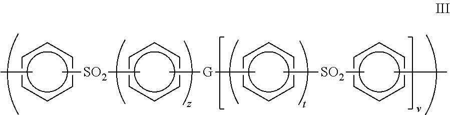
wherein m,r,s,t,v,w and z independently represent zero or a positive integer, E and E′ independently represent an oxygen or a sulphur atom or a direct link, G represents an oxygen or sulphur atom, a direct link or a —O-Ph-O—moiety where Ph represents a phenyl group and Ar is selected from one of the following moieties (i)**, (i) to (iv) which is bonded via one or more of its phenyl moieties to adjacent moieties

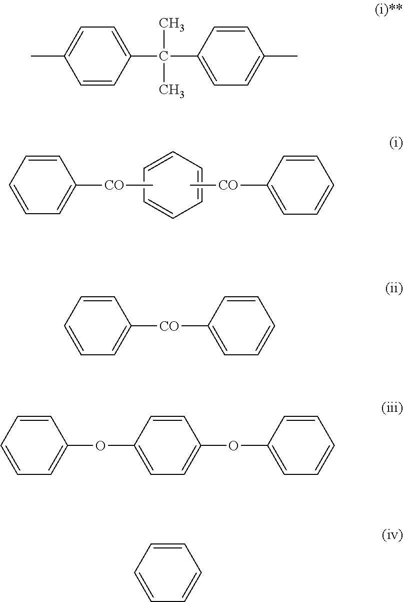

5. An enclosure according to claim 4, wherein said polymeric material comprises a repeat unit of formula (XX) 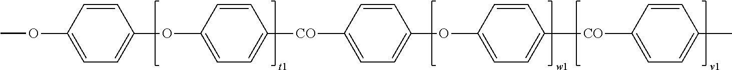
where t1, and w1 independently represent 0 or 1 and v1 represents 0, 1 or 2.

6. An enclosure according to claim 4, wherein said polymeric material is selected from polyetheretherketone, polyetherketone, polyetherketoneetherketoneketone and polyetherketoneketone.

7. An enclosure according to claim 4, wherein said polymeric material comprises polyetheretherketone.

8. An enclosure according to claim 1, wherein the first region is a component of an enclosure wall which is arranged to substantially fully enclose an internal volume in which components of an implantable device may be contained.

9. An enclosure according to claim 1, where said first region has a thickness of at least 1 μm and of less than 200 μm.

10. An enclosure according to claim 1, wherein said first region comprises a metal, ceramic or plastics material.

11. An enclosure according to claim 1, wherein said first region comprises titanium.

12. An enclosure according to claim 1, wherein said first and second regions are defined by first and second layers of the materials described.

13. An enclosure according to claim 1, which includes a third region inwardly of the second region and inwardly of the first region, wherein said third region is arranged to electronically insulate components which may, in use, be contained within an internal volume of the enclosure.

14. An enclosure according to claim 13 , wherein said third region is a component of an enclosure wall of the enclosure, wherein said enclosure wall is arranged to substantially fully enclose an internal volume in which components of an implantable device may be contained.

15. An enclosure according to claim 13, wherein said third region has a thickness of at least 10 μm and a thickness of less than 500 μm.

16. An enclosure according to claim 1, which includes a communications area comprising a region of the enclosure which is arranged to more readily transmit electromagnetic radiation between a position outside the housing and electronic components which may be provided within the housing in use.

17. An enclosure according to claim 16, wherein the ratio of the thickness of the first region in the region of the communications area to the thickness outside the area is in the range 0.2 to 0.8 and the ratio of the thickness of the second region in the region of the communications area to the thickness in areas of the second region which surround the communications area is at least 1.05.

18. An enclosure according to claim 1, wherein said first region comprises titanium and said second region comprises polyetheretherketone.

19. An enclosure according to claim 18, said enclosure including a communications window which comprises a region of the enclosure which is arranged to more readily transmit electromagnetic radiation compared to a region of said enclosure around the communications window, wherein said enclosure includes a communications device for communicating information to a position outside the enclosure.

20. An enclosure according to claim 13, wherein said first region comprises titanium, said second region comprises polyetheretherketone and said third region comprises polyetheretherketone.

21. (canceled)

22. A structure according to claim 2428, wherein said first region comprises titanium, said second region comprises a polymeric material of formula (XX), and said third region comprises a polymeric material of formula (XX).

23. An implantable device comprising an enclosure according to claim 1.

24. (canceled)

25. A method of treating a condition of a human body comprising:
selecting an implantable device according to claim 23;
implanting the implantable device into the human body, wherein the implantable device is arranged to apply a stimulus to the body to treat the condition.

26. A method of making an implantable device according to claim 23, the method comprising selecting an enclosure which comprises a first region having a water vapour transmission rate of less than 1g.m⁻².d⁻¹ and a second region outside the first region, wherein said second region comprises a second material having a Notched Izod Impact Strength of at least 1.0 KJ.m⁻², and associating means for applying a stimulus to a part of a human body with the enclosure.

27. A structure according to claim 28, wherein said first region comprises titanium, said second region comprises polyetheretherketone, and said third region comprises polyetheretherketone.

28. A layered structure comprising:
a first region having a water vapour transmission rate of less than 1g.m⁻².d⁻¹;
a second region having a second material having a Notched Izod Impact Strength of at least 1.0 KJ.m⁻²; and
a third region inwardly of the second region and inwardly of the first region, wherein said third region is arranged to electronically insulate components which may, in use, be contained within an internal volume of the enclosure.
